(19) United States
(12) Patent Application Publication
(10) Pub. No.: US 2019/0269915 A1
(43) Pub. Date: Sep. 5, 2019

(54) ARTIFICIAL EYE AND VISUAL AID DEVICE

(71) Applicant: Seiko Epson Corporation, Tokyo (JP)

(72) Inventor: Hiroaki HOSOMI, Minowa-MACHI (JP)

(21) Appl. No.: 16/346,426

(22) PCT Filed: Oct. 10, 2017
(86) PCT No.: PCT/JP2017/036647

§ 371 Date: Apr. 30, 2019

(30) Foreign Application Priority Data
Nov. 2, 2016 (JP) 2016-214915
Nov. 2, 2016 (JP) 2016-214916

(51) Int. Cl. A61N 1/36; A61N 1/372

(52) U.S. Cl. CPC A61N 1/36046; H04N 5/2253; A61N 1/37217

(57) ABSTRACT

An image can be easily transmitted to an artificial retina in a contactless manner.

An artificial eye 10 has an imaging unit 16 that captures an image in a viewing direction of a user 11 who is a living body, a light emitting unit 19 that emits light indicating the captured image of the imaging unit 16, and an artificial retina 20 that is implanted in the user 11, receives laser light from the light emitting unit 19, and transmits information corresponding to light-receiving results to optic nerves 45 of the user 11.

### TECHNICAL FIELD

The invention relates to an artificial eye and a visual aid device.

### BACKGROUND ART

Researches have been performed to make an eye visible in even patients with retinal pigmentary degeneration. In some countries, transplantations have been made in a plurality of patients under public approval.

Among them, a configuration in which an electrode tip that functions as an artificial retina is embedded in a patient's eye, and an image captured by a camera built in a glasses type terminal is transmitted to the electrode tip by utilizing electromagnetic induction is suggested (for example, refer to PTL 1).

### CITATION LIST

### Patent Literature

PTL 1: US-A-2013/0144360

### SUMMARY OF INVENTION

### Technical Problem

However, in the related-art configurations, it is necessary to perform surgery on a patient's eye, and it is necessary for a patient to wear a glasses type terminal. That is, irrespective of performing the surgery on the patient's eye, use cannot be made unless an external device is not used outside a human body. Additionally, although the electromagnetic induction is a technique suitable for non-contact power transmission, this is not suitable for transmission of high-density information, including an image or the like due to restrictions or the like of transmission speed. Additionally, since the electromagnetic induction requires magnets and coils, there are restrictions on downsizing of devices.

Thus, an object of the invention is to enable an image to be easily transmitted to an artificial retina in a contactless manner. Additionally, another object of the invention is to cause a living body to recognize an image without using an external device.

### Solution to Problem

[Application Example 1] In order to achieve the above object, an artificial eye of the present application example includes an imaging unit that captures an image in a viewing direction of a living body; a light emitting unit that emits light indicating the captured image of the imaging unit; and an artificial retina that is implanted in the living body, receives the light from the light emitting unit, and transmits information corresponding to light-receiving results to optic nerves of the living body.

According to the present application example, restrictions or the like of transmission speed can be solved compared to a case where electromagnetic induction is used for the transmission of the information to the artificial retina. Additionally, magnets, coils and the like are not used, which is advantageous to the downsizing of the device correspondingly. Therefore, an image can be transmitted to the artificial retina in a contactless manner.

[Application Example 2] In the artificial eye described in the above application example, the artificial retina is disposed along a curved face of the retina on an inner face of the retina.

According to the present application example, the surgery of implanting the artificial retina becomes easy compared to a configuration in which a plurality of signal lines extend from the artificial retina and are connected to the optic nerves.

[Application Example 3] In the artificial eye described in the above application example, the light emitting unit extracts a profile of the captured image and emits light indicating the profile.

According to the present application example, it is easy to recognize the outer shape of an object by reducing the amount of information to be transmitted to the living body.

[Application Example 4] In the artificial eye described in the above application example, the light emitting unit has a light source that emits laser light, and a light scanning unit that performs scanning with the laser light in two-dimensional directions.

According to the present application example, compared to a case where light other than the laser light is used, downsizing is easy, and adjustment adapted to the living body is easy.

[Application Example 5] In the artificial eye described in the above application example, the imaging unit and the light emitting unit are provided in a wearable device to be worn on the living body without being implanted in the living body, and the light emitting unit passes the light through at least any one of a pupil or a crystalline lens of the living body and causes the light to reach the artificial retina.

According to the present application example, an image can be transmitted to the artificial retina in a contactless manner while utilizing the light quantity regulating function of the pupil, the lens function of the crystalline lens, or the like.

[Application Example 6] In the artificial eye described in the above application example, the imaging unit and the light emitting unit are implanted in the living body, and the light emitting unit passes the light through a tissue of the living body and causes the light to reach the artificial retina.

According to the present application example, the information can be easily transmitted to the artificial retina by the configuration in which the imaging unit and the light emitting unit are implanted in the living body.

[Application Example 7] The artificial eye described in the above application example further includes a reflecting member that is implanted in the living body, and reflects the light from the light emitting unit toward the artificial retina.

According to the present application example, the degree of freedom of arrangement of the light emitting unit is improved, and the light emitting unit is easily disposed at a position where the light emitting unit is easily disposed in the living body.

[Application Example 8] A visual aid device of the present application example includes an imaging unit that captures an image in a viewing direction of a living body; a light emitting unit that emits light indicating the captured image of the imaging unit; and an artificial retina that is implanted in any one of eyes of the living body, receives the light from the light emitting unit, and transmits information corresponding to light-receiving results to optic nerves of the living body.

According to the present application example, the vision of the living body is aided, and an image can be transmitted to the artificial retina in a contactless manner. Additionally, as the artificial retina receives the light indicating an image that has passed through at least any one of a pupil and a crystalline lens of the living body, the light quantity regulating function of the pupil or the lens function of the crystalline lens can be utilized.

[Application Example 9] An artificial eye of the present application example includes an imaging unit that is implanted in a living body and captures an image in a viewing direction of the living body; an optic nerve stimulating unit that is implanted in the living body and stimulates optic nerves of the living body; and an image transmission unit that is implanted in the living body and transmits the captured image of the imaging unit to the optic nerve stimulating unit.

According to the present application example, the imaging unit, the optic nerve stimulating unit, and the image transmission unit can be implanted in the living body, and the living body can be caused to recognize an image without using an external device.

[Application Example 10] In the artificial eye described in the above application example 9, the image transmission unit extracts a profile of the captured image and emits light indicating the profile.

According to the present application example, it is easy to recognize the outer shape of an object by reducing the amount of information to be transmitted to the living body.

[Application Example 11] In the artificial eye described in the above Application Examples 9 and 10, the imaging unit is implanted in one eye of eyes of the living body and the optic nerve stimulating unit is implanted in the other eye.

According to the present application example, it is possible to dispose the imaging unit and the optic nerve stimulating unit by effectively using a space between both the eyes of the living body.

[Application Example 12] In the artificial eye described in any one of the above Application Examples 9 to 11, the optic nerve stimulating unit is an artificial retina in contact with a retina of the living body.

According to the present application example, it is possible to transmit a stimulus to the optic nerves by utilizing the artificial retina.

[Application Example 13] In the artificial eye described in the above Application Example 12, the image transmission unit passes the light indicating the captured image through a tissue of the living body and causes the light to reach the artificial retina, and the artificial retina receives the light and applies a stimulus corresponding to light-receiving results to the optic nerves.

According to the present application example, light is used for transmission of an image. Thus, restrictions or the like of transmission speed can be solved compared to a case where electromagnetic induction is used for the transmission of the information. Additionally, magnets, coils and the like are not used, which is advantageous to the downsizing of the device correspondingly.

[Application Example 14] In the artificial eye described in the above Application Example 13, the artificial retina is disposed along a curved face of the retina on an inner face of the retina.

According to the present application example, a surgical operation in which the signal lines are connected to the optic nerves becomes unnecessary, and the surgery of implanting the artificial retina becomes easy.

[Application Example 15] The artificial eye described in the above Application Example 13 or 14 further includes a reflecting member that is implanted in the living body, and reflects the light toward the artificial retina from the image transmission unit.

According to the present application example, the degree of freedom of arrangement of the image transmission unit is improved, and the image transmission unit is easily disposed at a position where the light emitting unit is easily disposed in the living body.

[Application Example 16] In the artificial eye described in any one of the above Application Examples 13 to 15, the image transmission unit has a light source that emits laser light, and a light scanning unit that performs scanning with the laser light in two-dimensional directions.

According to the present application example, compared to a case where light other than the laser light is used, downsizing is easy, and adjustment adapted to the living body is easy.

[Application Example 17] A visual aid device of the present application example includes an imaging unit that is implanted in a living body and captures an image in a viewing direction of the living body; an optic nerve stimulating unit that is implanted in the living body and stimulates optic nerves of the living body; and an image transmission unit that is implanted in any one of eyes of the living body and transmits the captured image of the imaging unit to the optic nerve stimulating unit.

According to the present application example, the imaging unit, the optic nerve stimulating unit, and the image transmission unit can be implanted in the living body, and the vision of the living body can be aided without using an external device.

### BRIEF DESCRIPTION OF THE DRAWINGS

FIG. 1 is a view illustrating a mode of use of an artificial eye related to a first embodiment of the invention.

FIG. 2 is a block diagram illustrating a functional configuration of the artificial eye.

FIG. 3 is a view illustrating a light emitting unit and an artificial retina together with peripheral components.

FIG. 4 is a view of the light emitting unit as seen from a side.

FIG. 5 is a view illustrating a state where a wearable device is worn by a user with long hair.

FIG. 6 is a view illustrating an artificial retina of a second embodiment together with peripheral components.

FIG. 7 is a view illustrating a mode of use of an artificial eye related to a third embodiment of the invention.

FIG. 8 is a block diagram illustrating a functional configuration of the artificial eye.

FIG. 9 is a view illustrating a layout structure of the artificial eye.

FIG. 10 is a view illustrating a layout structure of an artificial eye related to a fourth embodiment.

FIG. 11 is a view illustrating a layout structure of an artificial eye related to a fifth embodiment.

FIG. 12 is a view illustrating a layout structure of an artificial eye related to a sixth embodiment.

FIG. 13 is a view illustrating an example of a visual aid device provided in a right eye.

### DESCRIPTION OF EMBODIMENTS

Hereinafter, preferred embodiments of the invention will be described in detail, referring to the drawings. In addition, the embodiments to be described below do not limit the contents of the invention described in the claims. Additionally, all components to be described below are not necessarily essential components of the invention.

### First Embodiment

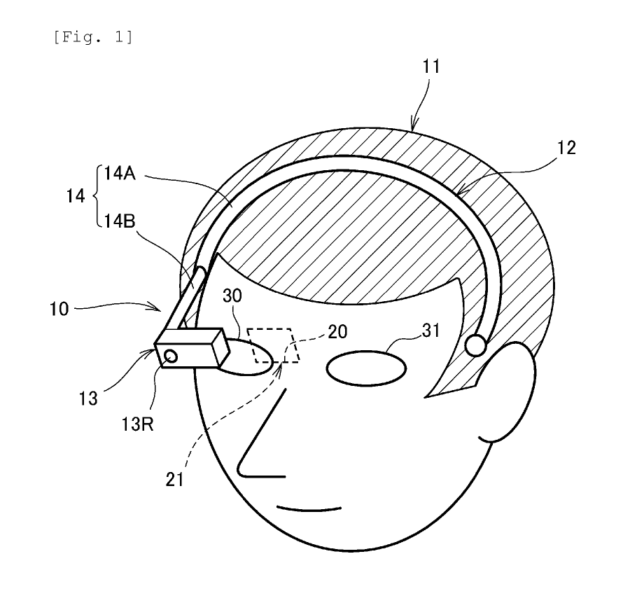

FIG. 1 is a view illustrating a mode of use of an artificial eye related to a first embodiment.

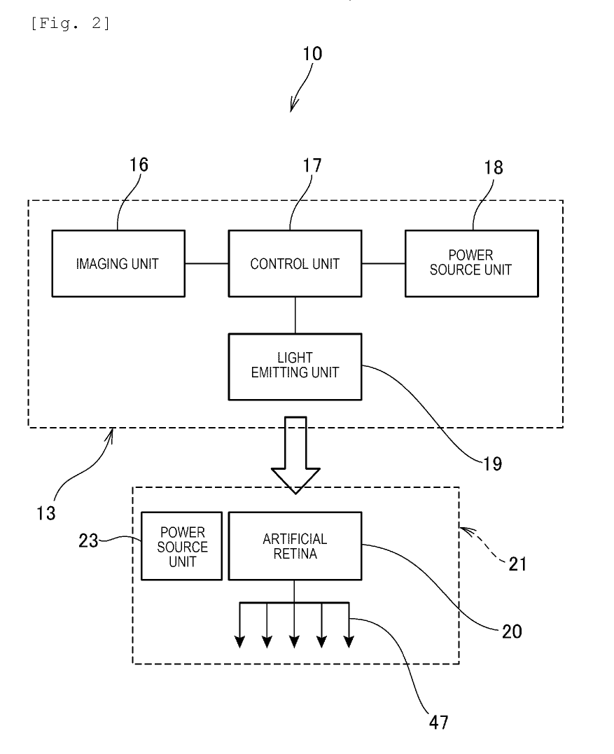

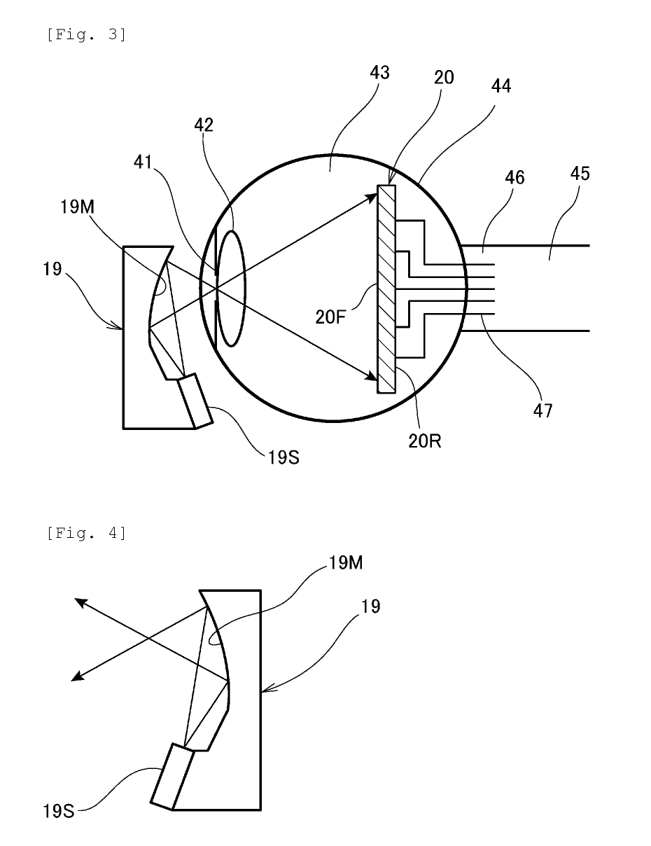

An artificial eye 10 includes a wearable device 12 that functions as an extracorporeal device to be worn outside a body of a user 11, and an implant 21 that includes an artificial retina 20 and is an intracorporeal device to be worn inside the body of the user 11 (FIG. 2). The artificial retina 20 is a retina alternative that stimulates optic nerves 45 of the user 11 (refer to FIG. 3) to artificially reproduce vision. The user 11 is, for example, a visually impaired person who does not have eyesight (vision) due to retinal pigmentary degeneration or the like. Although a case where the artificial retina 20 has been implanted in a right eye 30 is exemplified in the present embodiment, the artificial retina 20 may be implanted in a left eye 31. Additionally, although the case of the artificial eye 10 to be applied to humans will be described, the artificial eye 10 maybe applied to other living bodies, such as dogs and cats.

The wearable device 12 is easily attachable and detachable by the user 11, and is worn by the user 11 when the artificial eye 10 is used. The wearable device 12 includes a body section 13 that has an imaging function, and an information transmission function of transmitting a captured image to the artificial retina 20, and a supporting member 14 that supports the body section 13.

The supporting member 14 has a band section 14A that is freely attachable to and detachable from the head of the user 11, and an arm section 14B that supports the body section 13 such that the body section 13 is disposed at a position spaced apart from the band section 14A. In addition, all directions (a forward direction, a rearward direction, an upward-downward direction, and a leftward-rightward direction) to be described are respective directions as seen from the user 11 when the wearable device 12 is worn.

The band section 14A is formed of elastic materials, such as plastic materials, which has elasticity, pinches a region that includes left and right temples of the user 11 with an elastic force, and causes the supporting member 14 to be held on the head of the user 11. The band section 14A is formed, for example, in a shape in which the external appearance of the band section 14A is similar to a headband (hair band) that is a hair accessory.

The arm section 14B extends forward from the band section 14A, and performs supporting such that the body section 13 is disposed ahead of the right eye 30 of the user. Since the body section 13 is disposed ahead of the right eye 30 of the user 11, an image (video) of the right eye 30 in a viewing direction can be easily taken in by a lens 13R provided on a front face of the body section 13. Additionally, a light emitting unit 19 (refer to FIG. 2) is provided on a back face of the body section 13, and is capable of emitting light from the light emitting unit 19 to the right eye 30 in a state where there is no shading between the body section 13 and the right eye 30.

FIG. 2 is a block diagram illustrating a functional configuration of the artificial eye 10.

The body section 13 includes an imaging unit 16, a control unit 17, a power source unit 18, and the light emitting unit 19.

The imaging unit 16 is an imaging device that has an imaging element of a plurality of pixels, for example, is a CCD image sensor or a CMOS image sensor. The imaging unit 16 captures an image of the user 11 in the viewing direction via the lens 13R of the body section 13, and outputs image data corresponding to the captured image. Accordingly, the imaging unit 16 is capable of capturing an image equivalent to an image to be viewed by the right eye 30 of the user 11.

The image in the viewing direction captured by the imaging unit 16 includes an image in front of the right eye 30. However, limitation to the image in front of the right eye 30 is not made. For example, an image including some or all of a viewing range of general right eyes may be included, or an image including some or all of a viewing range of both general eyes may be included. Additionally, an image in a range exceeding a viewing range of humans may also be included. In short, an image desired to be perceived by the user 11 may be included. Additionally, the lens 13R may be configured such that at least one of the direction and focal point thereof is variable under the control of the control unit 17.

The control unit 17 is constituted of a processor that performs arithmetic processing, and a peripheral circuit, and is constituted of, for example, an image processing CPU, and a memory that stores a control program. As the image processing CPU executes the control program, the control unit 17 performs optimization processing for emitting light in the light emitting unit 19 with respect to the image data output from the imaging unit 16, and transmits signals subjected to the optimization processing to the light emitting unit 19.

The optimization processing includes profile extraction processing in which the profile of the captured image is extracted from the image data of the imaging unit 16. Then, the control unit 17 converts image data showing the profile of the captured image extracted by profile extraction processing into predetermined signals suitable for driving of the light emitting unit 19 to output the signals. Accordingly, the light emitting unit 19 emits light indicating the profile of the captured image, and the profile is easily perceived by the user 11 by the artificial retina 20. In this case, since the amount of information to be transmitted to the user 11 is reduced compared to a case where the light indicating the entire captured image is emitted, this also allows the user 11 to easily recognize an object. In addition, the profile extraction processing may be omitted.

The power source unit 18 is a device that supplies operating power to respective units (the imaging unit 16, the control unit 17, and the light emitting unit 19) of the body section 13, and has, for example, a button battery to be used for a wrist watch or a small-sized digital camera. In addition, a solar battery that receives surrounding light to generate electric power may be used as well as the button battery.

The light emitting unit 19 is a device that emits the light indicating the captured image toward the artificial retina 20 under the control of the control unit 17.

FIG. 3 is a view illustrating the light emitting unit 19 and the artificial retina 20 with peripheral components. FIG. 3 illustrates an internal structure of the right eye 30 as seen from a side of the user 11, reference sign 41 represents a pupil of the right eye 30, reference sign 42 represents a crystalline lens, reference sign 43 represents a vitreous body, reference sign 44 represents a retina, reference sign 45 represents optic nerves, and reference sign 46 represents optic disc.

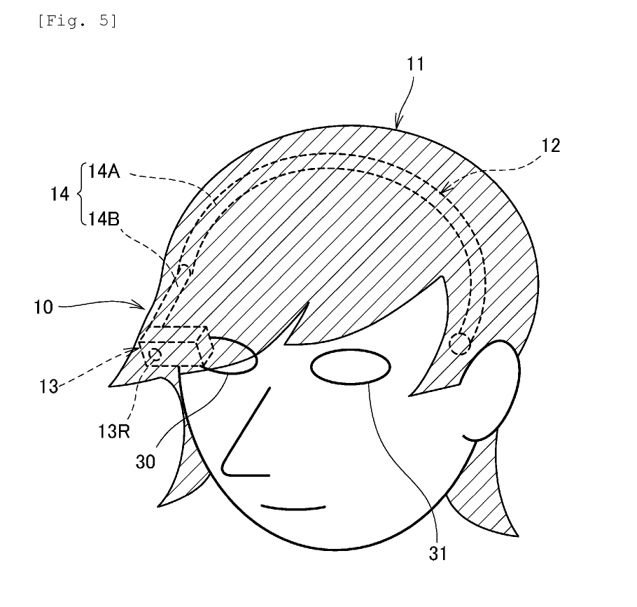

FIG. 4 is a view of the light emitting unit 19 as seen from the side.

The light emitting unit 19 has a light scanning unit (also referred to as an optical scanner) 19S that has a light source for emitting laser light built therein and changes the direction of the laser light, and a projection mirror 19M that reflects the laser light from the light scanning unit 19S.

As illustrated in FIG. 4, the light scanning unit 19S is disposed under the projection mirror 19M, reflects the laser light (indicated by arrows in FIG. 4) toward the projection mirror 19M, and performs scanning with laser light in two-dimensional directions (for example, the upward-downward direction and the leftward-rightward direction). The light scanning unit 19S is, for example, a MEMS mirror, and the small-sized light scanning unit 19S is obtained by using the MEMS mirror.

As illustrated in FIG. 3, the projection mirror 19M of the present embodiment is formed in a shape that is recessed forward, and is disposed in front of the right eye 30. The projection mirror 19M reflects the laser light from the light scanning unit 19S rearward, thereby causing the laser light to enter the pupil 41 of the right eye 30. Accordingly, the laser light passes through the pupil 41 and the crystalline lens 42, and enters the artificial retina 20.

By utilizing the pupil 41 and the crystalline lens 42 in this way, the laser light enters the right eye 30 from the outside. Thus, the laser light can be caused to enter the right eye 30 simply and reliably and to reach the artificial retina 20.

In the present configuration, as the light scanning unit 19S performs scanning with single laser light in the two-dimensional directions, light (that is, image light) indicating a two-dimensional image corresponding to the captured image is projected on the artificial retina 20. Accordingly, the light emitting unit 19 functions as an image transmission unit that transmits the captured image to the artificial retina 20 in a contactless manner.

By using the laser light with which the scanning is performed in the two-dimensional directions, the magnitude or the like of the image light can be easily adjusted, and adjustment according to the user 11 is possible. Additionally, downsizing is easier compared to a case where other light sources are used.

Additionally, since the light scanning unit 19S and the projection mirror 19M are disposed so as to be vertically distributed, the length (equivalent to depth) of the light emitting unit 19 in the forward-rearward direction is suppressed, and the separation distance between the light scanning unit 19S and the projection mirror 19M is easily secured. For this reason, the distance from the light emitting unit 19 to the artificial retina 20 is easily secured without increasing the separation distance between the light emitting unit 19 and the right eye 30, and the light emitting unit 19 can be disposed in close proximity to the right eye 30.

In addition, the arrangement of the light scanning unit 19S and the projection mirror 19M is not limited to the above arrangement and can be appropriately changed. Additionally, the projection mirror 19M may be omitted, and the light from the light scanning unit 19S may be caused to directly enter the pupil 41. Additionally, the light scanning unit 19S may not be limited to the MEMS mirror, and may have, for example, a configuration using a polygon mirror, a galvanometer mirror, or the like.

In the present embodiment, since the laser light is caused to enter the right eye 30 from the outside by utilizing the pupil 41 and the crystalline lens 42, the light quantity regulating function of the pupil 41 and the lens function of the crystalline lens 42 can be utilized. In addition, a flexible arm that bends the position of the body section 13 in finely adjustable manner is used for the arm section 14B that supports the body section 13, and the position of the body section 13 can be easily adjusted depending on the position of the right eye 30.

FIG. 5 is a view illustrating a state where the wearable device 12 is worn by the user 11 with long hair.

As illustrated in FIG. 5, in a case where the hair of the user 11 is long, the band section 14A of the wearable device 12 can be covered with the hair, and the arm section 14B and the body section 13 can be covered with forelock. In other words, the wearable device 12 can be made invisible from the outside by adopting the hairstyle in which the band section 14A, the arm section 14B, and the body section 13 are covered.

As illustrated in FIGS. 2 and 3, the implant 21, which is an intracorporeal device to be worn inside the body of the user 11, has the artificial retina 20 and a power source unit 23.

The power source unit 23 is a device that supplies operating power to the artificial retina 20, and has a battery, such as a solar battery or a biological battery. The solar battery is a device that generates electric power by utilizing the light (a portion of solar light or laser light) that has entered the right eye 30, and the biological battery is a device that generates electric power by utilizing bioenergy, such as the body temperature of the user 11. In addition, the invention is not limited to the batteries having the power generation function, and for example, a configuration in which a high-capacity primary battery is built, a configuration in which a secondary battery is built and is wirelessly charged from the outside, or the like may be adopted.

The artificial retina 20 has a plurality of photoelectric conversion elements (also referred to as light receiving elements), and sends electrical signals created by the photoelectric conversion elements, which have received the laser light, to the optic nerves 45 of the right eye 30. The artificial retina 20 of the present embodiment is formed in a flat plate shape that extends in the upward-downward direction in a side view, and the plurality of photoelectric conversion elements are arranged on a face 20F on the pupil 41 side to constitute a photoelectric conversion element array.

Additionally, a plurality of signal lines 47 are connected to a face 20R of the artificial retina 20 opposite to the pupil 41. The plurality of signal lines 47 are connected to the optic disc 46 equivalent to a junction between the optic nerves 45 and the retina 44, and are capable of transmitting information (equivalent to an electrical stimulus) to the optic nerves 45 associated with a light-receiving area in advance.

The artificial retina 20 sends the electrical signals created by the respective photoelectric conversion elements to the optic nerves 45 via the signal lines 47 corresponding to a light-receiving position for the laser light. Accordingly, the artificial retina 20 functions as an optic nerve stimulating unit that stimulates the optic nerves 45.

The positions of pixels of the imaging unit 16 and the positions of pixels to be drawn on the artificial retina 20 correspond to each other such that the user 11 is caused to recognize a correct image. In addition, the artificial retina 20 adjusts the electrical signals to have voltage values and current values that are suitable for stimulating the optic nerves 45 and outputs the adjusted electrical signals. Additionally, the artificial retina 20 may have a configuration in which the plurality of photoelectric conversion elements are grouped for each region, and the electrical signals are sent to the signal lines 47 in units of groups.

Since the artificial retina 20 has the signal lines 47 between the artificial retina 20 and the retina 44, the artificial retina 20 can be disposed apart from the retina 44. For this reason, the artificial retina 20 can be disposed without being dependent on the shapes or the like of retinas 44 of individual users 11, and the artificial retina 20 common to the users 11 from children to adults is easily applied. In addition, a structure in which the artificial retina 20 is integrated with the above power source unit 23 may be adopted.

As described above, the artificial eye 10 of the present embodiment has the imaging unit 16 that captures an image in the viewing direction of the user 11 who is a living body, the light emitting unit 19 that emits light indicating the captured image of the imaging unit 16, and the artificial retina 20 that is implanted in the user 11, receives the laser light from the light emitting unit 19, and transmits information corresponding to light-receiving results to the optic nerves 45 of the user 11. Accordingly, restrictions or the like of transmission speed can be solved compared to a case where electromagnetic induction is used for the transmission of the information to the artificial retina 20. Additionally, magnets, coils and the like are not used, which is advantageous to the downsizing of the device correspondingly. Therefore, an image can be transmitted to the artificial retina 20 in a contactless manner.

Additionally, since the light emitting unit 19 extracts the profile of the captured image and emits the light indicating the profile, it is easy to recognize the outer shape of an object by reducing the amount of information to be transmitted to the user 11.

Additionally, since the light emitting unit 19 has a light source that emits the laser light, and the light scanning unit 19S that performs scanning with the laser light in the two-dimensional directions, it is easy to realize the downsizing compared to a case where light other than the laser light is used. Moreover, by adjusting the scanning range of the laser light, the image light can be passed through the pupil 41 of the user 11 and emitted with a size or the like optimal for the artificial retina 20. Therefore, adjustment adapted to the user 11 is easy.

Additionally, the imaging unit 16 and the light emitting unit 19 are provided in the wearable device 12 to be worn by the user 11 without implanted in the user 11, and the light emitting unit 19 passes light through the pupil 41 and the crystalline lens 42 of the user 11 to project the light on the artificial retina 20. Accordingly, light can be easily caused to reach the artificial retina 20 within the user 11 from the outside. In addition, this configuration can be applied to a case where at least any one of the pupil 41 and the crystalline lens 42 is normal and light indicating an image enters an eye.

Additionally, since the light emitting unit 19 passes the image light through the pupil 41 of the user 11 to cause the image light to reach the artificial retina 20, the information can be transmitted to the optic nerves 45 by utilizing the light quantity regulating function or the like of the pupil 41.

### Second Embodiment

The artificial eye 10 of the first embodiment has a configuration that the plurality of signal lines 47 extend from the flat plate-shaped artificial retina 20 and leads to the optic nerves 45, and requires a surgical operation in which the signal lines 47 are connected to the individual optic nerves 45. In an artificial eye 110 of the second embodiment, the surgical operation in which the signal lines 47 are connected to the individual optic nerves 45 is made unnecessary by disposing an artificial retina 120 along a curved face of the retina 44 on an inner face of the retina 44.

In addition, in the artificial eye 110, the components other than artificial retina 120 are the same as those of the first embodiment. In the following description, portions different from the first embodiment will be mainly described, the same portions will be designated by the same reference signs, and duplicate description will be omitted.

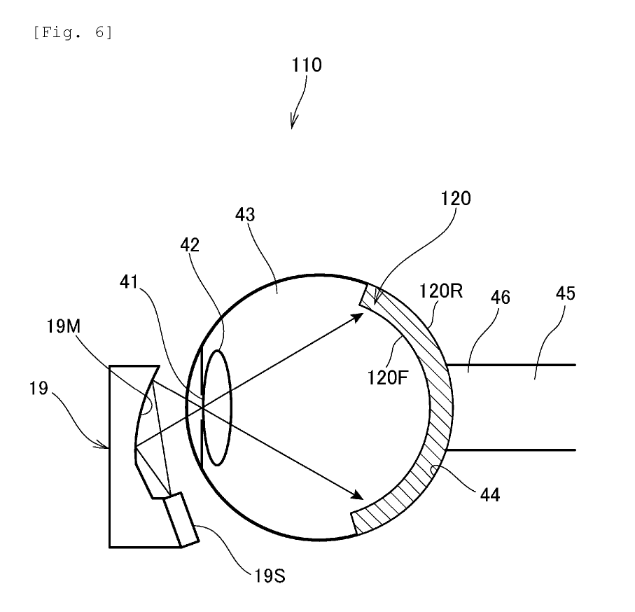

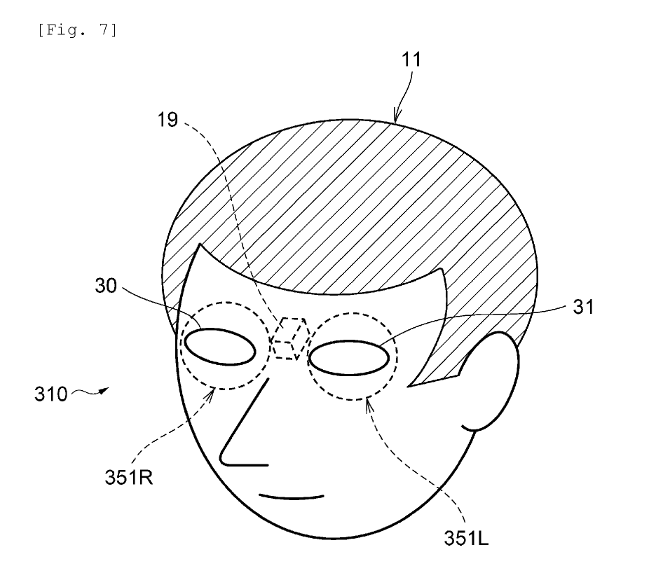

FIG. 6 is a view illustrating the artificial retina 120 of the second embodiment together with peripheral components.

As illustrated in FIG. 6, the artificial retina 120 of the artificial eye 110 is disposed along the curved face of the retina 44 on the inner face of a region having the optic disc 46 in the retina 44. That is, the artificial retina 120 is formed in a spherical shape along the inner face of the retina 44.

The plurality of photoelectric conversion elements are arranged on a concave face 120F, which is the face of the artificial retina 120 on the pupil 41 side, to constitute the photoelectric conversion element array. Additionally, electrodes corresponding to the photoelectric conversion elements, respectively, are arranged on a convex face 120R, which is the face of the artificial retina 120 opposite to the pupil 41, to constitute an electrode array.

The artificial retina 120 is implanted in the right eye 30 such that the convex face 120R is in contact with the inner face of the retina 44. The research of artificially reproducing vision is roughly and representatively classified into three types: a type in which the retina 44 is stimulated, a type in which the optic nerves 45 is stimulated, and a type in which the brain is directly stimulated. The artificial retina 120 of the second embodiment corresponds to a type in which the retina 44 is stimulated. That is, the electrode array is installed on the retina 44 to directly apply an electric stimulus to a retina ganglion cell layer, and thereby, information is transmitted to the optic nerves 45 via the optic disc 46, and is recognized (discovered as eyesight) as an image in the visual cortex of the brain of the user 11. In addition, the artificial retina 20 of the first embodiment is a type in which the optic nerves are stimulated.

In the artificial eye 110 of the second embodiment, since the curved artificial retina 120 suitable for each user 11 is adopted, the surgical operation in which the signal lines 47 are connected to the individual optic nerves 45 becomes unnecessary. Therefore, the surgery of implanting the artificial retina 120 become easier than that in the first embodiment. However, it is desirable to select any one of the first and second embodiments depending on the situation or the like of the user 11.

Additionally, the artificial eyes 10 and 110 of the first and second embodiments are applied to one eye (right eye 30) of the user 11 but may be applied to both the eyes 30 and 31 (the right eye 30 and the left eye 31). Stereoscopic viewing is enabled in a case where the artificial eyes are applied to both the eyes 30 and 31.

### Third Embodiment

In the first and second embodiments, it is necessary to wear the wearable device 12 when the user 11 uses the artificial eyes 10 and 110. In a third embodiment, the wearable device 12 is not required when the user 11 uses an artificial eye 310.

In the following description, portions different from the first embodiments will be mainly described, the same portions will be designated by the same reference signs, and duplicate description will be omitted. Additionally, all the directions (the forward direction, the rearward direction, the upward-downward direction, and the leftward-rightward direction) to be described are respective directions as seen from the user 11 that has worn the artificial eyes.

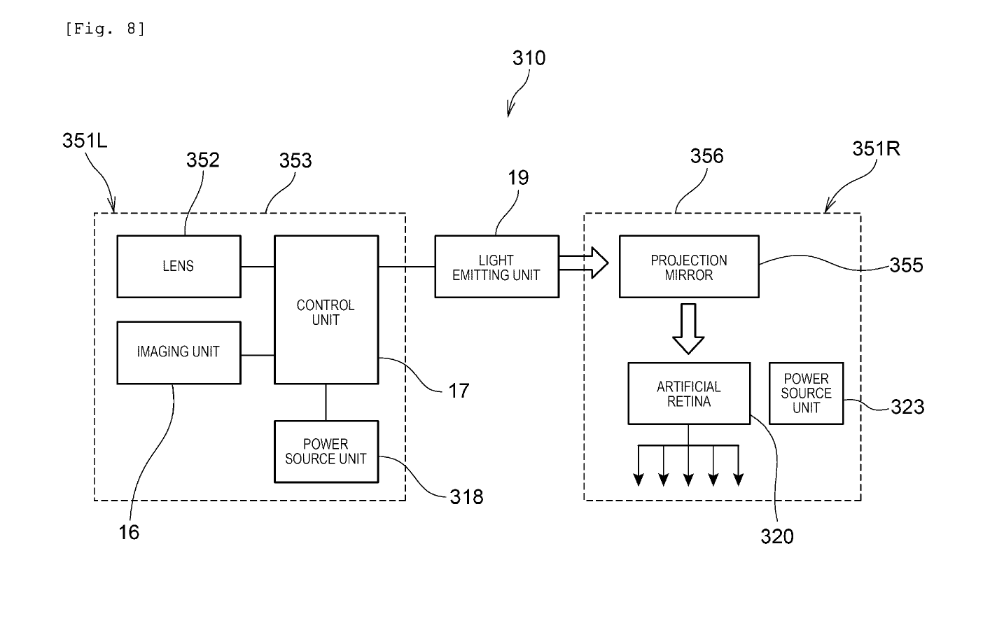

FIG. 7 is a view illustrating a mode of use of the artificial eye related to the third embodiment.

In the artificial eye 310, in addition to an artificial retina 320, components (the imaging unit 16, the control unit 17, and the light emitting unit 19) of the body section 13 of the wearable device 12 are implanted in the user 11. That is, all the components of the artificial eye 310 are implanted in the user 11.

In detail, the artificial eye 310 includes a first artificial eye 351L to be worn in the left eye 31 of the user 11, the light emitting unit 19, and a second artificial eye 351R to be worn in the right eye 30.

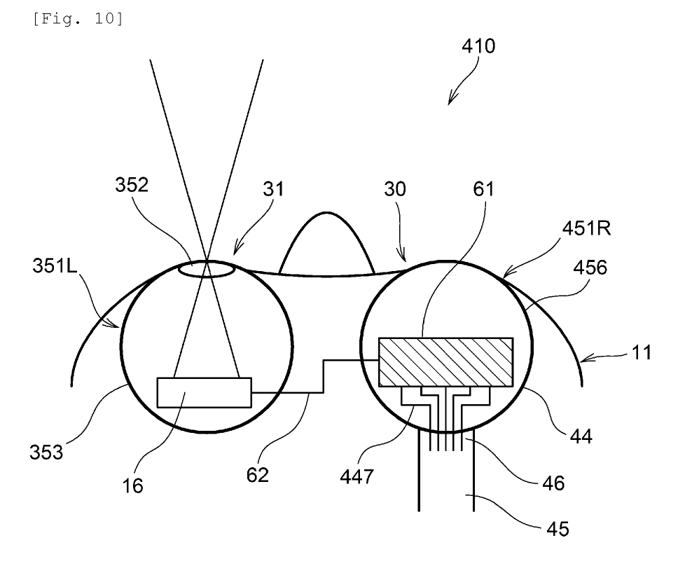

In the present embodiment, since the retinal cells and the optic nerves 45 (refer to FIG. 9) of the right eye 30 are used, at least the function of sensing the electrical stimulus needs to remain even if the user 11 cannot recognize light with the retina 44 (FIG. 9). In addition, optic nerves of the left eye 31 may not function at all.

All the respective units, such as the first artificial eye 351L, the second artificial eye 351R, and the light emitting unit 19, are implanted in the body of the user 11 by surgery. In addition, it is desirable that it is not known that these components are implanted inside the body of the user 11 from the external appearance depending on surgery that does not leave scars or makeup. Although the case of the artificial eye 310 to be applied to humans has been described in the present embodiment, the artificial eye 310 may be applied to other living bodies, such as dog and cats.

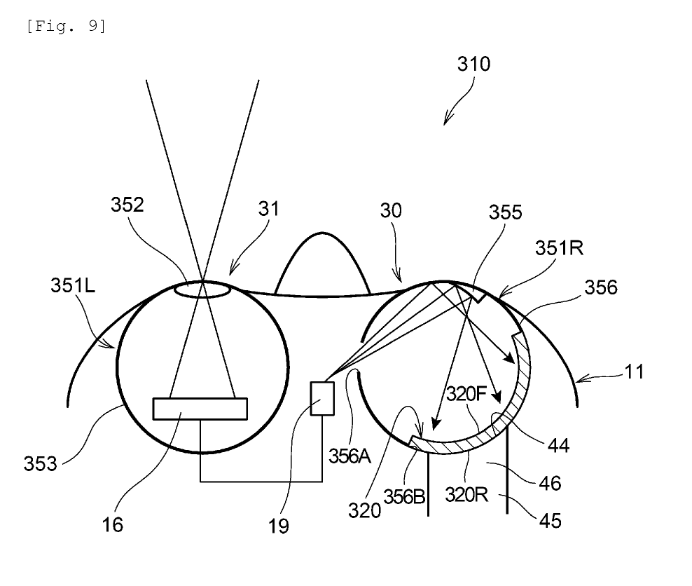

FIG. 8 is a block diagram illustrating a functional configuration of the artificial eye 310.

The first artificial eye 351L is disposed within the eyeball of the left eye 31. The first artificial eye 351L includes the imaging unit 16, the control unit 17, a power source unit 318, a lens 352, and a first artificial eye body 353 that supports these. As the first artificial eye body 353 is implanted in the left eye 31, the imaging unit 16 and the like are implanted in the left eye 31.

The second artificial eye 351R is disposed within the eyeball of the right eye 30. The second artificial eye 351R includes a projection mirror 355, an artificial retina 320, a power source unit 323, and a second artificial eye body 356 that supports these. As the second artificial eye body 356 is implanted in the right eye 30, the artificial retina 320 and the projection mirror 355 are implanted in the right eye 30.

The imaging unit 16 captures an image of the user 11 in the viewing direction via the lens 352, and outputs the image data corresponding to the captured image.

The lens 352 is disposed so as to be capable of taking the image of the user 11 in the viewing direction into the first artificial eye 351L. The lens 352 is a movable lens capable of changing any one of the direction and focal point thereof under the control of the control unit 17.

The image in the viewing direction captured by the imaging unit 16 includes an image in front of the left eye 31. However, the captured image is not limited to the image in front of the left eye 31. For example, an image including some or all of a viewing range of general left eyes may be included, or an image including some or all of a viewing range of both general eyes may be included. Additionally, an image in a range exceeding a viewing range of humans may also be included. In short, an image desired to be perceived by the user 11 may be included.

The control unit 17 transmits the signals subjected to the optimization processing similar to the above embodiment to the light emitting unit 19. Accordingly, the light emitting unit 19 emits light indicating the profile of the captured image, and the profile is easily perceived by the user 11 by the artificial retina 320. In this case, since the amount of information to be transmitted to the user 11 is reduced compared to a case where the light indicating the entire captured image is emitted, this also allows the user 11 to easily recognize an object. In addition, the profile extraction processing may be omitted.

The power source unit 318 is a device that supplies operating power to respective units (the imaging unit 16 and the control unit 17) of the first artificial eye 351L, the light emitting unit 19), and the like, and has, for example, a button battery to be used for a wrist watch or a small-sized digital camera. In addition, a solar battery that receives surrounding light to generate electric power may be used as well as the button battery.

The light emitting unit 19 is a device that emits the light corresponding to the captured image toward the second artificial eye 351R including the artificial retina 320 under the control of the control unit 17.

The first artificial eye body 353 is formed of a shielding material or the like which does not affect a living body tissue and which solar light or the like does not enter.

FIG. 9 is a view illustrating a layout structure of the artificial eye 310. In addition, FIG. 9 is a view schematically illustrating the inside of the head from above the user 11, reference sign 44 represents the retina, reference sign 45 represents the optic nerves, and reference sign 46 represents the optic disc equivalent to the junction between the optic nerves 45 and the retina 44.

The light emitting unit 19 is disposed between the first artificial eye 351L and the second artificial eye 351R, when being implanted in a region equivalent to between the left and right eyes 30 and 31 of the user 11.

Although illustration of the control unit 17 and the power source unit 318 is omitted, the control unit 17 and the power source unit 318 may be disposed within the first artificial eye 351L or the second artificial eye 351R, or may be disposed at the same position as that of the light emitting unit 19. Additionally, the power source unit 318, and the power source unit 323 of the artificial retina 320 may be common to each other, or may be separate from each other.

As illustrated in FIG. 9, the light emitting unit 19 emits the laser light toward the second artificial eye 351R (projection mirror 355 to be described below) at the right oblique front in a top view. That is, the light emitting unit 19 passes the laser light through the living body tissue (a subcutaneous tissue, a blood vessel, or the like) and emits the laser light to the second artificial eye 351R.

The projection mirror 19M is formed in a shape recessed toward the left oblique rear and reflects the laser light from the light scanning unit 19S toward the second artificial eye 351R.

As illustrated in FIGS. 8 and 9, the second artificial eye 351R includes the projection mirror 355 that functions as a reflecting member that reflects the laser light from the light emitting unit 19 toward the artificial retina 320, and the second artificial eye body 356 that supports the projection mirror 355.

As illustrated in FIG. 9, the second artificial eye body 356 has an opening 356A to which a region corresponding to the emission light of the light emitting unit 19 opens, and an opening 356B to which a region corresponding to the artificial retina 320 opens. By virtue of the opening 356A, the laser light reflected by the projection mirror 19M enters the second artificial eye 351R without being blocked by the second artificial eye body 356 and is projected on the artificial retina 320.

In addition, the second artificial eye body 356 is formed of the shielding material or the like which does not affect the living body tissue and which the solar light or the like does not enter. Additionally, the artificial retina 320 may be configured such that a portion thereof is supported by the second artificial eye body 356.

In the present configuration, as the light scanning unit 19S performs scanning with the single laser light in the two-dimensional directions, the light (that is, the image light) indicating the two-dimensional image corresponding to the captured image is projected on the artificial retina 320. Accordingly, the light emitting unit 19 functions as the image transmission unit that transmits the captured image to the artificial retina 320 in a contactless manner.

By using the laser light with which the scanning is performed in the two-dimensional directions, the magnitude or the like of the image light can be easily adjusted, and the adjustment according to the user 11 is possible. Additionally, the downsizing is easier compared to a case where other light sources are used.

Additionally, since the light scanning unit 19S and the projection mirror 19M are disposed so as to be vertically distributed, the length (equivalent to depth) of the light emitting unit 19 in the forward-rearward direction is suppressed, and the separation distance between the light scanning unit 19S and the projection mirror 19M is easily secured. For this reason, the degree of freedom of arrangement is improved such as easily securing the distance from the light emitting unit 19 to the artificial retina 320 without increasing the separation distance between the light emitting unit 19 and the right eye 30, and disposing the light emitting unit 19 in close proximity to the right eye 30.

In addition, the arrangement of the light scanning unit 19S and the projection mirror 19M is not limited to the above arrangement and can be appropriately changed. Additionally, the projection mirror 19M may be omitted, and the light from the light scanning unit 19S may be caused to directly enter the opening 356A of the second artificial eye 351R.

The projection mirror 355 within the second artificial eye 351R is disposed in a radiation range of the laser light that enters from the opening 356A, and reflects the laser light to projects the laser light on the artificial retina 320. The shape and arrangement of the projection mirror 355 may be changed depending on the position or the like of the light emitting unit 19.

The artificial retina 320 functions as the optic nerve stimulating unit that stimulates the optic nerves 45. The artificial retina 320 has the plurality of photoelectric conversion elements (also referred to as the light receiving elements), and sends the electrical signals created by the photoelectric conversion elements, which have received the laser light, to the optic nerves 45 of the right eye 30. The artificial retina 320 of the present embodiment is disposed along the curved face of the retina 44 on the inner face of a region having the optic disc 46 in the retina 44. That is, the artificial retina 320 is formed in a spherical shape along the inner face of the retina 44.

The plurality of photoelectric conversion elements are arranged on a concave face 320F, which is the face of the artificial retina 320 on a light-receiving side, to constitute the photoelectric conversion element array. Additionally, the electrodes corresponding to the photoelectric conversion elements, respectively, are arranged on a convex face 320R, which is the face of the artificial retina 320 opposite to the concave face 320F, to constitute the electrode array.

The artificial retina 320 is implanted in the right eye 30 such that the convex face 320R is in contact with the inner face of the retina 44. In the research of artificially reproducing vision, the artificial retina 320 corresponds to a type in which the retina 44 is stimulated, and the electrode array is installed on the retina 44 to directly apply an electric stimulus to the retina ganglion cell layer. Accordingly, information is transmitted to the optic nerves 45 via the optic disc 46 and is recognized (discovered as eyesight) as an image in the visual cortex of the brain of the user 11.

The positions of pixels of the imaging unit 16 and the positions of pixels to be drawn on the artificial retina 320 correspond to each other such that the user 11 is caused to recognize a correct image. In addition, the artificial retina 320 adjusts and outputs the electrical signals to the voltage values and the current values that are suitable for stimulating the optic nerves 45. Additionally, the artificial retina 320 may have a configuration in which the plurality of photoelectric conversion elements are grouped for each region, and the optic nerves 45 are stimulated in units of groups.

In the present embodiment, the curved artificial retina 320 suitable for each user 11 is adopted. Thus, a surgical operation in which the signal lines are connected to the individual optic nerves 45 becomes unnecessary compared to a configuration in which the signal lines extend from the artificial retina and the signal lines are connected to the optic nerves 45. Therefore, the surgery of implanting the artificial retina 320 becomes easy.

In this way, in the present embodiment, a camera (equivalent to the imaging unit 16) is disposed at the left eye 31 of the user 11, a screen (equivalent to the artificial retina 320) is disposed at the right eye 30 of the user 11, and a projector (equivalent to the light emitting unit 19) is disposed between the left and right eyes 30 and 31 of the user 11.

Since the human's eyeballs are small, it is difficult to lay out both the imaging unit 16 and the artificial retinas 320 for stimulating the retina 44 in one eyeball. In consideration of this difficulty, in the present embodiment, attention is paid to the presence of two human eyes, and a configuration is adopted in which the imaging unit 16 is disposed in one eye of a pair of eyes and the artificial retina 320 is disposed in the other eye. In addition, it is possible to appropriately select whether the imaging unit 16 or the artificial retina 320 is disposed in any eye.

As long as various devices required for the artificial eye 310 of the present embodiment are downsized due to technological advances, all the devices may be implanted in one eye (the right eye 30 or the left eye 31). Additionally, the imaging unit 16 and the artificial retina 320 may be disposed in a pair in the left and right eyes 30 and 31. Stereoscopic viewing becomes possible if images of the left and right eyes 30 and 31 can be recognized.

As described above, the artificial eye 310 of the present embodiment includes the imaging unit 16 that is implanted in the user 11 who is a living body and captures an image in the viewing direction of the user 11, the artificial retina 320 that is implanted in the user 11 and has functions as the optic nerve stimulating unit that stimulates the optic nerves 45 of the user 11, and the light emitting unit 19 that is implanted in the user 11 and functions as the image transmission unit that transmits the captured image of the imaging unit 16 to the artificial retina 320. Accordingly, it is possible to implant the imaging unit 16, the optic nerve stimulating unit (artificial retina 320), and the image transmission unit (light emitting unit 19) in the user 11 and to cause the user 11 recognize an image without using an external device.

Additionally, since the camera (equivalent to the imaging unit 16) is disposed in one eye (left eye 31) of the user 11 and the screen (equivalent to the artificial retina 320) is disposed in the other eye (right eye 30) of the user 11, the respective members can be disposed by effectively using a space between both the eyes 30 and 31 of the user 11.

Additionally, since the artificial retina 320 in contact with the retina 44 of the user 11 is used as the optic nerve stimulating unit that stimulates the optic nerves 45, a stimulus can be transmitted to the optic nerves 45 by utilizing the artificial retina 320.

Moreover, since the artificial retina 320 is disposed along the curved face of the retina 44 on the inner face of the retina 44, the surgical operation in which the signal lines are connected to the optic nerves 45 becomes unnecessary. Therefore, the surgery of implanting the artificial retina 320 becomes easy.

Additionally, since the light emitting unit 19 that functions as the image transmission unit passes the light (laser light) indicating the captured image through the living body tissue of the user 11 and causes the light to reach the artificial retina 320, an image can be easily transmitted within the living body. Moreover, since light is used for the transmission of the image, transmission of high-density information, such as an image, is easy, the restrictions or the like of the transmission speed can be solved compared to a case where the electromagnetic induction or the like is used for the transmission of the information. Additionally, magnets, coils and the like are not used, which is advantageous to the downsizing of the device correspondingly. Additionally, an image can be transmitted to the artificial retina 320 in a contactless manner, and it is easy to suppress an influence on a human body.

Additionally, since the light emitting unit 19 extracts the profile of the captured image and emits the light indicating the profile, it is easy to recognize the outer shape of an object by reducing the amount of information to be transmitted to the user 11.

Additionally, since the projection mirror 355, which is implanted in the user 11 and functions as the reflecting member that reflects the light of the light emitting unit 19 toward the artificial retina 320, is included, the degree of freedom of arrangement of the light emitting unit 19 is improved, and the light emitting unit 19 is easily disposed at a position where the light emitting unit 19 is easily disposed within the living body.

Additionally, since the light emitting unit 19 has the light source that emits the laser light, and the light scanning unit 19S that performs scanning with the laser light in the two-dimensional directions, it is easy to realize the downsizing compared to a case where light other than the laser light is used. Moreover, by adjusting the scanning range of the laser light, the image light can be passed through the opening 356A and emitted with a size or the like optimal for the artificial retina 320. Therefore, the adjustment adapted to the user 11 is easy.

In addition, in the present embodiment, since all the components (also referred to as devices or electronic devices) that constitute the artificial eye 310 are implanted in the living body, it is desirable to use the biological battery, which generates power by utilizing the bioenergy, such as the body temperature of the user 11, for the supply of electric power to the respective components.

However, the present embodiment is not limited to using the biological battery, and the supply of electric power may be performed from the outside. In that case, if the supply of electric power is performed in a contactless manner by a technique, such as the electromagnetic induction, it is not necessary to expose electrodes to a human body surface.

### Fourth Embodiment

In the research of artificially reproducing vision, the artificial eye 310 of the third embodiment is the type in which the retina 44 is stimulated, and an artificial eye 410 of a fourth embodiment is a type in which the optic nerves 45 are stimulated.

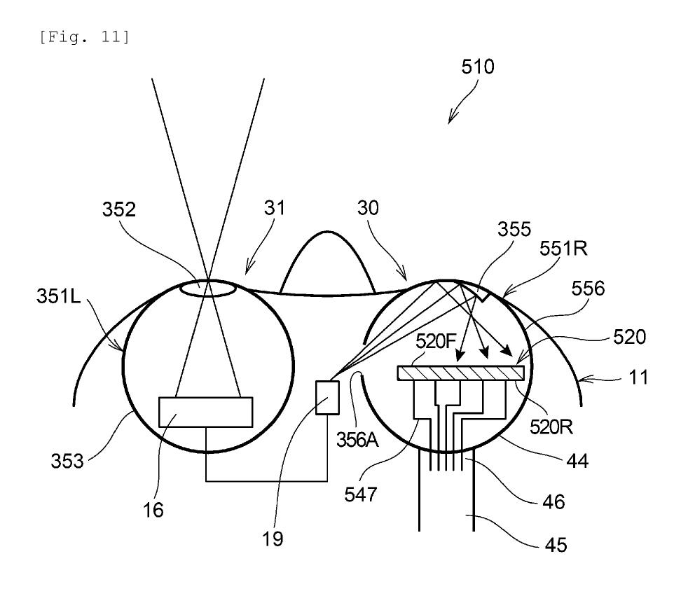

FIG. 10 is a view illustrating a layout structure of the artificial eye 410 related to the fourth embodiment. In addition, FIG. 10 is a view schematically illustrating the inside of a head from above the user 11.

The fourth embodiment is different from the third embodiment in that the optic nerve stimulating units that stimulate the optic nerves 45 are an electronic circuit 61 and signal lines (output lines) 447, and the image transmission unit that transmits the captured image to the electronic circuit 61 is a signal line (connection line) 62 that connects the imaging unit 16 and the electronic circuit 61 together.

In the following description, portions different from the embodiments described above will be mainly described, the same portions will be designated by the same reference signs, and duplicate description will be omitted.

As illustrated in FIG. 10, the artificial eye 410 includes the first artificial eye 351L to be worn in the left eye 31 of the user 11, a second artificial eye 451R to be worn in the right eye 30, and the signal line (connection line) 62.

The second artificial eye 451R is disposed within the eyeball of the right eye 30. The second artificial eye 351R includes the electronic circuit 61, the signal lines 447, and a second artificial eye body 456 that supports these.

An image captured by the imaging unit 16 of the first artificial eye 351L is subjected to optimization processing suitable for the driving of the electronic circuit 61 by the control unit 17 (not illustrated), and the signals subjected to the optimization processing are sent to the electronic circuit 61 through the signal line 62 which functions as the connection line that connects the imaging unit 16 and the electronic circuit 61 together. Similarly to the third embodiment, the optimization processing also includes the profile extraction processing in which the profile of the captured image is extracted from the image data of the imaging unit 16. For this reason, the image data showing the profile of the captured image is converted into predetermined signals suitable for driving of the electronic circuit 61 and the converted signals are output to the electronic circuit 61.

Here, since the signal line 62 is connected to the electronic circuit 61 within the second artificial eye 451R through the living body tissue from the inside of the first artificial eye 351L, the imaging unit 16 and the electronic circuit 61 are wire-connected together, which is advantageous to transmission of high-density information, including an image.

The electronic circuit 61 has the plurality of signal lines 447 that are output lines of the electronic circuit 61, and the signal lines 447 are connected to the optic disc 46 equivalent to the junction between the optic nerves 45 and the retina 44. Then, as the electronic circuit 61 transmits information (equivalent to an electrical stimulus) to the optic nerves 45 associated with individual pixels of the captured image in advance via the signal lines 447, the image captured by the imaging unit 16 is correctly recognized by the user 11.

The electronic circuit 61 can be a circuit that adjusts electrical signals to have voltage values and current values suitable to stimulate the optic nerves 45, and outputs the adjusted electrical signals, and can have, for example, a configuration equivalent to the electrode array of the artificial retina 320 of the above third embodiment, or a configuration to which an existing amplifying circuit or the like is applied. In addition, the electronic circuit 61 may have a function of grouping individual regions of the captured image and transmitting the electrical signals to the optic nerves 45 in units of groups.

Since the electronic circuit 61 is wire-connected to the optic nerves 45, contact portions between the signal lines 447 and the optic disc 46 are equivalent to locations where the optic nerves 45 are stimulated. The electrical signals output from the signal lines 447 are sent to the visual cortex of the brain of the user 11 via the optic nerves 45, and the user 11 is caused to recognize the electrical signals (discover eyesight) as an image.

Since the fourth embodiment has a configuration in which the plurality of signal lines 447 extend from the electronic circuit 61 and are connected to the optic nerves 45, the electronic circuit 61 can be disposed apart from the retina 44. For this reason, it is not necessary to form the electronic circuit 61 in a shape along the curved face of the retina 44, and simple shapes, such as a flat plate shape, can be applied. Accordingly, the electronic circuit 61 can be disposed without being dependent on the shapes or the like of retinas 44 of individual users 11, and the electronic circuit 61 common to the users 11 from children to adults is easily applied.

In addition, in the fourth embodiment, the surgical operation in which the signal lines 447 are connected to the individual optic nerves 45 is required. Thus, it is desirable to select any one of the third or fourth embodiment depending on the situation or the like of the user 11.

The present embodiment can also be said to have a structure in which a camera (equivalent to the imaging unit 16) is disposed at the left eye 31 of the user 11, and a screen (equivalent to the electronic circuit 61) is disposed at the right eye 30 of the user 11. For this reason, the respective members can be disposed by utilizing the space between both the eyes 30 and 31 of the users 11, and compared to a case where both the camera and the screen are implanted in one eye, the degrees of freedom of the sizes of the camera and the screen are high, and surgery is also easy.

Additionally, in the third embodiment, the captured image is transmitted by utilizing light (light emitting unit 19), whereas in the fourth embodiment, the captured image is transmitted by utilizing the electrical signals flowing through the signal line 62. A space for the signal line 62 can be saved compared to the light emitting unit 19, which is also advantageous to cost reduction.

Additionally, even in the fourth embodiment, all the components that constitute the artificial eye 410 are implanted in the living body. Thus, the same various kinds of effects as those in the third embodiment, such as allowing an image to be recognized without using an external device, can be obtained.

### Fifth Embodiment

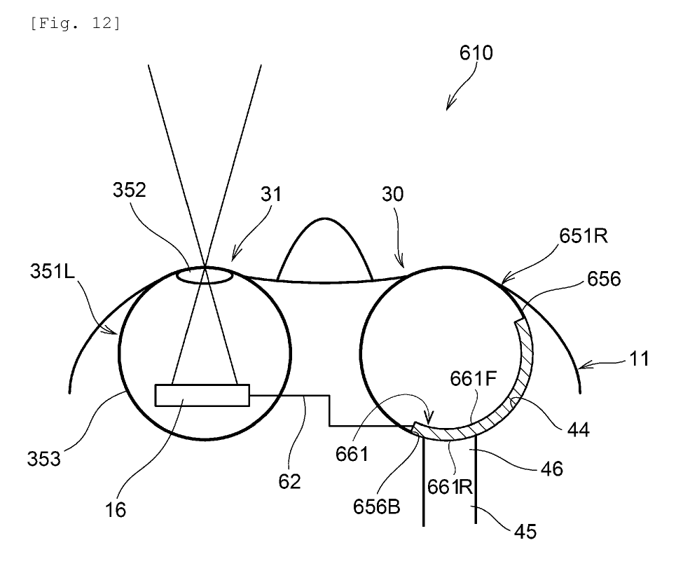

FIG. 11 is a view illustrating a layout structure of an artificial eye 510 related to a fifth embodiment. The fifth embodiment is the same as that of the artificial eye 310 of the third embodiment except that an artificial retina 520 and signal lines 547 that function as the optic nerve stimulating unit, and a second artificial eye body 556 are different.

As illustrated in FIG. 11, the artificial eye 510 includes the first artificial eye 351L to be worn in the left eye 31 of the user 11, the light emitting unit 19, and a second artificial eye 551R to be worn in the right eye 30.

The second artificial eye 551R includes the projection mirror 355, the artificial retina 520, the signal lines 547, the power source unit 323 (not illustrated), and the second artificial eye body 556 that supports these.

The second artificial eye body 556 has the opening 356A to which the region corresponding to the emission light of the light emitting unit 19 opens. By virtue of the opening 356A, the laser light reflected by the projection mirror 19M enters the second artificial eye 551R without being blocked by the second artificial eye body 556 and is projected on the artificial retina 520.

In addition, the second artificial eye body 556 is formed of the shielding material or the like which does not affect the living body tissue and which the solar light or the like does not enter.

The artificial retina 520 is formed in a flat plate shape that extends in the leftward-rightward direction in a top view, and the plurality of photoelectric conversion elements are arranged on a face 520F on the front side to constitute the photoelectric conversion element array. Additionally, the plurality of signal lines 547 are connected to a face 520R on a rear side. The plurality of signal lines 547 are connected to the optic disc 46 equivalent to a junction between the optic nerves 45 and the retina 44, and are capable of transmitting information (equivalent to an electrical stimulus) to the optic nerves 45 associated with the light-receiving area in advance. In addition, the positions of pixels of the imaging unit 16 and the positions of pixels to be drawn on the artificial retina 520 correspond to each other such that the user 11 is caused to recognize a correct image.

Since the artificial retina 520 has the signal lines 547 between the artificial retina 520 and the retina 44, the artificial retina 520 can be disposed apart from the retina 44. For this reason, the artificial retina 520 can be disposed without being dependent on the shapes or the like of retinas 44 of individual users 11, and the artificial retina 520 common to the users 11 from children to adults is easily applied. In addition, a structure in which the artificial retina 520 is integrated with the above power source unit 323 may be adopted.

### Sixth Embodiment

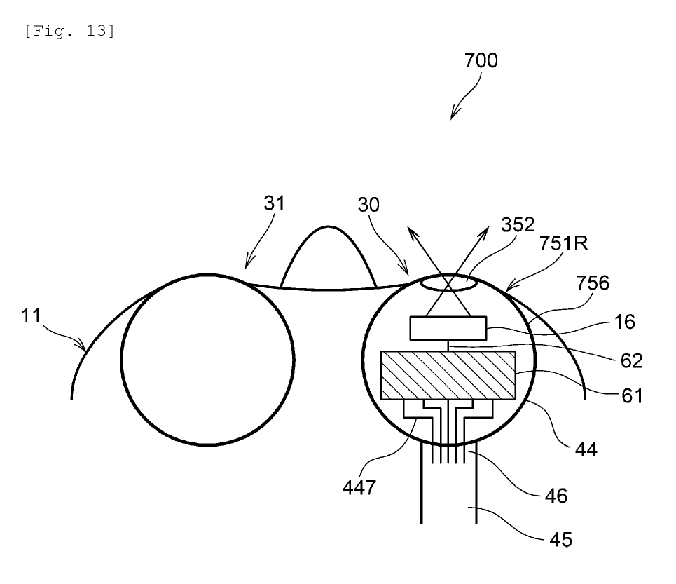

FIG. 12 is a view illustrating a layout structure of an artificial eye 610 related to a sixth embodiment. The sixth embodiment is the same as that of the artificial eye 410 of the fourth embodiment except that an electronic circuit 661 and a second artificial eye body 656 that function as the optic nerve stimulating unit are different.

As illustrated in FIG. 12, the artificial eye 610 includes the first artificial eye 351L to be worn in the left eye 31 of the user 11, and a second artificial eye 651R to be worn in the right eye 30. The second artificial eye 651R includes the electronic circuit 661, and the second artificial eye body that supports the electronic circuit. The second artificial eye body 656 has an opening 656B to which a region corresponding to the electronic circuit 661 opens.

The electronic circuit 661 is disposed along the curved face of the retina 44 on the inner face of a region including the optic disc 46 in the retina 44. That is, the electronic circuit 661 is formed in a spherical shape along the inner face of the retina 44 and is implanted in the right eye 30 so as to be in contact with the inner face of the retina 44.

In the electronic circuit 661, electrodes are arranged on a convex face 661R in contact with the retina 44 to constitute the electrode array, and each electrode array is installed to directly apply an electric stimulus to the retina ganglion cell layer. Accordingly, information is transmitted to the optic nerves 45 via the optic disc 46 and is recognized (discovered as eyesight) as an image in the visual cortex of the brain of the user 11. In addition, the positions of pixels of the imaging unit 16 and the positions of pixels to be drawn on the electronic circuit 661 correspond to each other such that the user 11 is caused to recognize a correct image.

In the present embodiment, the curved electronic circuit 661 suitable for each user 11 is adopted. Thus, the surgical operation in which the signal lines 447 are connected to the individual optic nerves 45 becomes unnecessary compared to a configuration (refer to FIG. 10) in which the signal lines 447 extend from the electronic circuit 61 and this signal lines 447 are connected to the optic nerves 45. Therefore, the surgery of implanting the electronic circuit 661 becomes easy.

The plurality of photoelectric conversion elements are arranged on a concave face 661F, which is the face of the electronic circuit 661 on the pupil 41 side, to constitute the photoelectric conversion element array.

In the above-described respective embodiments, a difference between the artificial retinas 20, 120, and 320, and the electronic circuits 61 and 661 is that the artificial retinas input the captured image depending on the light of the light emitting unit 19, whereas the electronic circuits input the captured image depending on the electrical signals from the imaging unit 16.

In the present specification, the artificial retinas 20, 120, and 320 and the electronic circuits 61 and 661 are described in a mutually distinguished manner for convenience depending on whether the light of the light emitting unit 19 is utilized. However, if a device that electrically stimulates nerve cells or nerve fibers remaining in the retina 44 to artificially recover vision is defined as the “artificial retina”, both the artificial retinas 20, 120, and 320 and the electronic circuits 61 and 661 correspond to the artificial retina.

Additionally, in the above-described first embodiment, a case where the wearable device 12 is configured as a hair band type has been described. However, the wearable device maybe configured as a glasses type, a goggles type, or a monocle type. In short, the wearable device 12 may be configured to be disposed in the vicinity of the eyes of the user 11.

Additionally, in the above-described respective embodiments, in a case where components (for example, the imaging unit 16, the control unit 17, the light emitting unit 19, the power source unit 318, the artificial retina 20, and the electronic circuit 61) that constitute an artificial eye is implanted in the living body, the components may be implanted in locations other than a face. For example, if the imaging unit 16 is buried in a hand, it is possible to view an image in a direction in which the hand is directed, and characteristics exceeding the related-art human's capacity are easily obtained.

Additionally, the artificial eyes 310 to 610 can be applied to a technique relating to cyborg in which various devices are implanted in a human body, and it is possible to develop various variations exceeding related-art human's capacities.

Additionally, in the artificial eyes of the above-described third to sixth embodiments, at least any one of the pupil and the crystalline lens of the user 11 is normal, and in a case where light indicating an image in the viewing direction enters an eye, at least any one of the pupil and the crystalline lens may be utilized instead of the lens 352. In this case, the light quantity regulating function of the pupil, the lens function the crystalline lens, or the like can be used.

In addition, in a case where there is a defect in any one of the pupil and the crystalline lens, a living body portion having the defect may be substituted with an artificial alternative.

### (Visual Aid Device)

In the above-described respective embodiments, the artificial eyes 10 to 610 that apply eyesight to the user 11 who is a visually impaired person hardly having eyesight (vision) have been described. The invention is not limited to this, and may be applied to a visual aid device that aids the vision of the user 11, such as a weak eyesight person.

As the visual aid device, it is possible to realize a configuration that is implanted in any one of the eyes 30 and 31 of the user 11 and aids the vision of the user 11. For example, the visual aid device is provided in the right eye 30 of the user 11 in a case where the left eye 31 of the user 11 has eyesight.

FIG. 13 is a view illustrating an example of a visual aid device 700 provided in the right eye 30. This visual aid device 700 includes a second artificial eye 751R to be worn in the right eye 30. The second artificial eye 751R is provided with all the components (the lens 352, the imaging unit 16, the electronic circuit 61, and the like) that constitute the artificial eye 410 of the fourth embodiment, and a second artificial eye body 756 that supports these components. As the second artificial eye 751R is implanted in the right eye 30 in this way, the respective units are implanted in the right eye 30.

By virtue of the second artificial eye 751R, an image in the viewing direction of the right eye 30 can be perceived by the user 11. According to this configuration, the stereoscopic viewing is enabled by the left eye 31 having eyesight and the visual aid device 700 implanted in the right eye 30.

In addition, the visual aid device 700 may have a configuration including the components that constitute the artificial eye 310 of the third embodiment.

Additionally, in a case where at least any one of the pupil 41 and the crystalline lens 42 of the user 11 is normal and light indicating an image enters an eye, the visual aid device can be realized by the same configuration as the artificial eyes 10 and 110 illustrated in FIGS. 1 to 6.

In addition, in the above-described artificial eyes 10 and 110 and the visual aid device, in a case where there is a defect in any one of the pupil 41 and the crystalline lens 42, a living body portion having the defect may be substituted with an artificial alternative. Additionally, in a case where neither the pupil 41 nor the crystalline lens 42 is normal, both the pupil 41 and the crystalline lens 42 may be substituted with artificial alternatives.

In the above-described artificial eyes 10 and 110 and the visual aid device, the case of the light indicating the image that has passed through the pupil 41 and the crystalline lens 42 has been described as an example of the light indicating the image that enters the living body from the outside of the living body. However, the invention is not limited to this. By adopting locations other than an eye as the positions of the artificial retinas 20 and 120, it is also possible to cause the light indicating the image entering the living body in other positions to enter the artificial retinas 20 and 120 and aid vision.

In addition, the above-described respective embodiments show one aspect of the invention, and modifications and applications are arbitrarily possible within the scope of the invention.

### REFERENCE SIGNS LIST

10: artificial eye (visual aid device)

11: user (living body)

12: wearable device

13: body section

13R: lens

14: supporting member

16: imaging unit

17: control unit

18, 23: power source unit

19: light emitting unit (image transmission unit)

19S: light scanning unit

19M: projection mirror

20: artificial retina (optic nerve stimulating unit)

21: implant

30: right eye

31: left eye

41: pupil

42: crystalline lens

43: vitreous body

44: retina

45: optic nerves

46: optic disc

47: signal line

351L: first artificial eye

351R: second artificial eye

353: first artificial eye body

355: projection mirror (reflecting member)

356: second artificial eye body

356A, 356B: opening

1-8. (canceled)

9. An artificial eye comprising:
an imaging unit that captures an image in a viewing direction of a living body of a user;
a light emitting unit that emits light indicating the captured image of the imaging unit; and
an artificial retina that is implanted in the living body, receives the light from the light emitting unit, and transmits information corresponding to light-receiving results to optic nerves of the living body,
wherein the imaging unit and the light emitting unit are provided in a wearable device to be worn on the living body without being implanted in the living body,
wherein the wearable device is configured to be invisible from an outside, and
wherein the light emitting unit passes the light through at least any one of a pupil or a crystalline lens of the living body and causes the light to reach the artificial retina.

10. The artificial eye according to claim 9,
wherein the artificial retina is disposed along a curved face of the retina on an inner face of the retina.

11. The artificial eye according to claim 9,
wherein the light emitting unit extracts a profile of the captured image and emits light indicating the profile.

12. The artificial eye according to claim 9,
wherein the light emitting unit has a light source that emits laser light, and a light scanning unit that performs scanning with the laser light in two-dimensional directions.

13. The artificial eye according to claim 9,
wherein the wearable device includes a body section including the imaging unit and the light emitting unit and an arm section that supports the body section, and
wherein as hair of the user covers the wearable device, the wearable device is configured to be invisible from the outside.

14. An artificial eye comprising:
an imaging unit that is implanted in one eye of both eyes of a living body and captures an image in a viewing direction of the living body;
an optic nerve stimulating unit that is implanted in the other eye of both the eyes of the living body and stimulates optic nerves of the living body; and
an image transmission unit that is implanted in the living body and transmits the captured image of the imaging unit to the optic nerve stimulating unit.

15. The artificial eye according to claim 14,
wherein the image transmission unit extracts a profile of the captured image and emits light indicating the profile.

16. The artificial eye according to claim 14,
wherein the optic nerve stimulating unit is an artificial retina in contact with a retina of the living body.

17. A visual aid device comprising:
a wearable device having an imaging unit that captures an image in a viewing direction of a living body of a user; and
an artificial retina that is implanted in an eye of the living body and transmits the captured image of the imaging unit to optic nerves of the living body,
wherein the wearable device is configured to be invisible from an outside.
